(19) United States
(12) Patent Application Publication
(10) Pub. No.: US 2022/0211313 A1
(43) Pub. Date: Jul. 7, 2022

(54) DRY ELECTROENCEPHALOGRAPHIC ELECTRODE

(71) Applicant: National Central University, Taoyuan City (TW)

(72) Inventors: Po-Lei Lee, Taoyuan City (TW); Kuo-Kai Shyu, Taoyuan City (TW)

(73) Assignee: National Central University, Taoyuan City (TW)

(21) Appl. No.: 17/564,696

(22) Filed: Dec. 29, 2021

(30) Foreign Application Priority Data
Jan. 4, 2021 (TW) 110100009

(51) Int. Cl. A61B 5/293; A61B 5/263

(52) U.S. Cl. CPC A61B 5/293; A61B 5/263

(57) ABSTRACT

A dry electroencephalographic (EEG) electrode includes a sleeve; an electrode protection member, where a plurality of first protective protrusions distributed in a comb shape are disposed on an annular plane of the electrode protection member; and a metal electrode, sandwiched between the sleeve and the electrode protection member, and including: an electrode connector, formed on and electrically connected to one plane of the metal electrode, and disposed in a hollow center of the sleeve; and a plurality of metal probes, electrically connected to another plane of the metal electrode. In a case that the dry EEG electrode is not pressed, ends of the probe extension portions of the plurality of metal probes protrude further relative to ends of the plurality of first protective protrusions of the electrode protection member, and in a case that the dry EEG electrode is pressed, the ends of the probe extension portions of the plurality of metal probes are flush with the ends of the plurality of first protective protrusions of the electrode protection member.

### CROSS-REFERENCE TO RELATED APPLICATIONS

This application claims priority to Taiwan Patent Application No. 110100009, filed Jan. 4, 2021, which is hereby incorporated by reference.

### TECHNICAL FIELD

The present invention relates to the field of medical devices, and in particular, to a dry electroencephalographic (EEG) electrode.

### BACKGROUND

Dry EEG electrodes are widely used at present, and will become the mainstream of EEG electrodes used in the future. However, most of the existing dry EEG electrodes are made with biocompatible metal probes for contact with skin and data measurement. In order that metal probes can achieve good measurement results through close contact, rigid metal probes are usually used, so that the metal probes can pass through hairs and slightly press on the scalp (or biological epidermis), to achieve good contact signal quality.

To perform dry EEG measurement without causing discomfort, improvements have been made as can be seen in many earlier patents. U.S. Patent Laid-open No. US20180092599A1 discloses a design using a multi-arm electrode headgear, where a dry EEG electrode is fixed to an elastic cap. The design uses metal arms, yet the metal arms have the problem of insufficient elasticity for a relatively distant EEG electrode position, and the dry arms cannot be firmly fixed on the scalp due to the pushing of hairs.

U.S. Patent Laid-open No. US20190000338A1 discloses a structure of a dry electrode that can be fine-tuned on the scalp, where the dry electrode may be moved to a relatively appropriate position through an adjustment slide rail. However, metal probe structures are still used as electrode probes, and there is a safety concern about an external impact, for example, the metal probe structure pierces the scalp.

U.S. Pat. No. 6,381,481B1 discloses an EEG headgear structure with a sleeve, where the sleeve is disposed at an appropriate position of the headgear and has an electrode placed therein, but does not mention how the electrode avoids blocking of the hair and protection between electrode probes and the scalp.

Taiwan Patent No. TW I638670 discloses a structure with a spring disposed inside a probe. However, there is no protective structure for an electrode or any protective protrusion. Taiwan Patent Laid-open No. TW 201714576 discloses a structure including an electrode, probes, and a shell. However, there is no protective structure for the electrode or any protective protrusion.

Most existing dry electrodes use metal probes for direct contact with the scalp, and therefore, do not have a function of protecting the scalp of a user from being impacted by an external force, which may cause the scalp of the user to be injured when being impacted by the external force. The existing dry electrodes also do not have a protection device to avoid the metal probe from contact with another charged device, resulting in a safe concern.

### SUMMARY

In view of the above problems, an objective of the present invention is to provide a dry EEG electrode, where a protection structure used in combination with a metal probe is adopted. The protection structure can protect an organism from being hurt by a probe impacted by an external force and piercing the scalp during dry EEG electrode measurement.

According to an aspect of the present invention, a dry EEG electrode is provided, including: a sleeve with a hollow center;

an electrode protection member, being hollow annular, where a plurality of first protective protrusions distributed in a comb shape are disposed on an annular plane of the electrode protection member; and

a metal electrode, sandwiched between the sleeve and the electrode protection member, including:
an electrode connector, formed on and electrically connected to one plane of the metal electrode, and disposed in the hollow center of the sleeve; and
a plurality of metal probes, distributed in a comb shape and electrically connected to another plane of the metal electrode, wherein each metal probe includes a probe fixing portion, a probe extension portion, and a spring, wherein the probe fixing portion is disposed on the other plane of the metal electrode, the spring is disposed in a hollow interior of the probe fixing portion, the probe extension portion is disposed in the hollow interior of the probe fixing portion, the probe extension portion squeezes the spring, and the spring produces a reaction force on the probe extension portion, which enables the probe extension portion to extend and contract relative to the probe fixing portion, wherein

in a case that the dry EEG electrode is not pressed, ends of the probe extension portions of the plurality of metal probes protrude further relative to ends of the plurality of first protective protrusions of the electrode protection member, and in a case that the dry EEG electrode is pressed, the ends of the probe extension portions of the plurality of metal probes are flush with the ends of the plurality of first protective protrusions of the electrode protection member.

According to an aspect of the present invention, the electrode connector of the metal electrode is electrically connected to a power connector in a magnetic attraction manner, so that the dry EEG electrode rotates in a horizontal direction relative to the power connector.

According to an aspect of the present invention, the sleeve and the electrode protection member are bonded with an adhesive, fastened with a screw, or engaged and fixed with a latch.

According to an aspect of the present invention, a plurality of second protective protrusions alternately distributed with the metal probes are disposed on the other plane of the metal electrode, and ends of the plurality of second protective protrusions are flush with the ends of the plurality of first protective protrusions.

According to an aspect of the present invention, the electrode protection member is made from rubber, biocompatible plastics, or an insulation material.

### BRIEF DESCRIPTION OF THE DRAWINGS

FIG. 1 shows a dry EEG electrode according to a first embodiment of the present invention, where FIG. 1(A) is an oblique view of the dry EEG electrode, and FIG. 1(B) is a side view of the dry EEG electrode;

FIG. 2 is an exploded view of the dry EEG electrode according to the first embodiment of the present invention;

FIG. 3 is a sectional view of a metal probe according to the first embodiment of the present invention; and

FIG. 4 is an oblique view of a dry EEG electrode according to a second embodiment of the present invention.

### DETAILED DESCRIPTION

To make a person of ordinary skill in the art further understand the present invention, preferred embodiments of the present invention are exemplified in the following descriptions, and with reference to the accompanying drawings, the components of the present invention and their expected effects are described in detail.

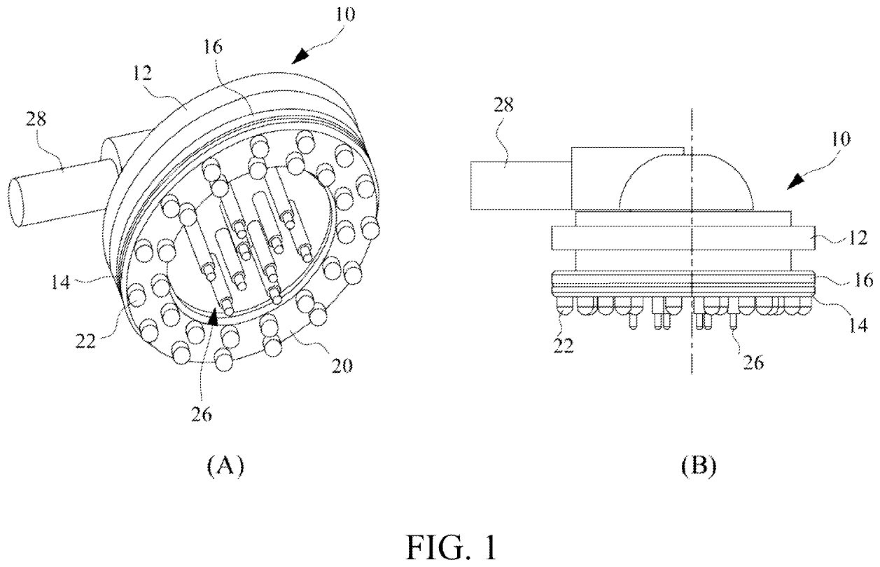

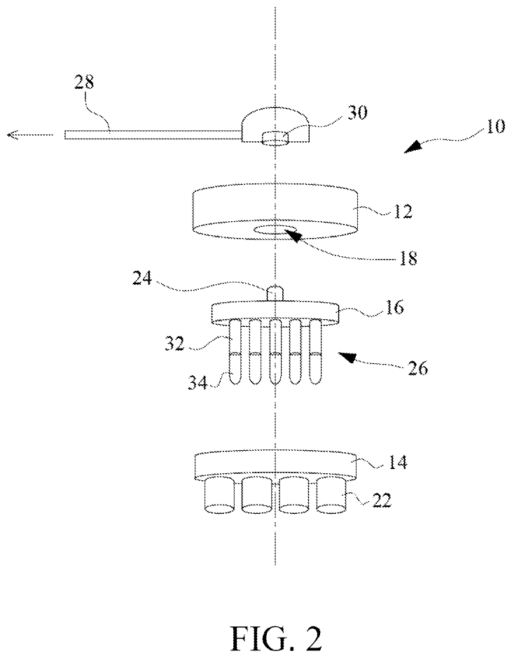

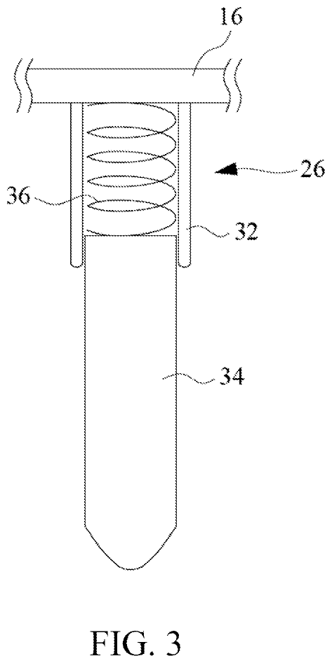

FIG. 1 shows a dry EEG electrode according to a first embodiment of the present invention, where FIG. 1(A) is an oblique view of the dry EEG electrode, and FIG. 1(B) is a side view of the dry EEG electrode. FIG. 2 is an exploded view of the dry EEG electrode according to the first embodiment of the present invention. FIG. 3 is a sectional view of a metal probe according to the first embodiment of the present invention.

In a first embodiment, a dry EEG electrode 10 includes a sleeve 12, an electrode protection member 14, and a metal electrode 16. The sleeve 12 includes a hollow center 18. The electrode protection member 14 has a hollow annular shape, where a plurality of first protective protrusions 22 distributed in a comb shape are disposed on an annular plane 20 of the electrode protection member 14. The electrode protection member 14 and the first protective protrusions 22 thereof are made from rubber, biocompatible plastics, or an insulation material. The annular electrode protection member 14 surrounds metal probes 26 (which will be described later) of the metal electrode 16, to avoid the metal probes 26 from contact with another charged device, thereby providing a safer electrical operation of the dry EEG electrode 10.

The metal electrode 16 is sandwiched between the sleeve 12 and the electrode protection member 14, and the sleeve 12 and the electrode protection member 14 are bonded with an adhesive, fastened with a screw, or engaged and fixed with a latch. The metal electrode 16 includes an electrode connector 24 and a plurality of metal probes 26.

The electrode connector 24 is formed on and electrically connected to one plane of the metal electrode 16, and is disposed in the hollow center 18 of the sleeve 12. The electrode connector 24 of the metal electrode 16 is electrically connected to a power connector 30 (which is shown in FIG. 2) of an external wire 28 in a magnetic attraction manner. Since the electrical connection is implemented in the magnetic attraction manner, the dry EEG electrode 10 rotates in a horizontal direction relative to the power connector 30 of the external wire 28.

The plurality of metal probes 26 distributed in a comb shape are electrically connected to another plane of the metal electrode 16. The metal probes 26 are distributed in a comb shape and pass through hair, and cooperates with the design that the dry EEG electrode 10 rotates in a horizontal direction relative to the power connector 30, to push the hair aside and smoothly touch the scalp.

Each metal probe 26 includes a probe fixing portion 32, a probe extension portion 34, and a spring 36. The probe fixing portion 32 is disposed on the other plane of the metal electrode 16, the spring 36 is disposed in a hollow interior of the probe fixing portion 32, the probe extension portion 34 is disposed in the hollow interior of the probe fixing portion 32, the probe extension portion 34 squeezes the spring 36, and the spring 36 produces a reaction force on the probe extension portion 34, which enables the probe extension portion 34 to extend and contract relative to the probe fixing portion 32. Since the metal probes 26 are extendable, the dry EEG electrode 10 provides the buffer function to the scalp, so that the dry EEG electrode 10 can conform to the ups and downs of the head shape during contact with the scalp.

In a case that the dry EEG electrode 10 is not pressed, ends of the probe extension portions 34 of the plurality of metal probes 26 protrude further relative to ends of the plurality of first protective protrusions 22 of the electrode protection member 14, and in a case that the dry EEG electrode 10 is pressed, the ends of the probe extension portions 34 of the plurality of metal probes 26 are flush with the ends of the plurality of first protective protrusions 22 of the electrode protection member 14. Such a protection structure may provide support when the dry EEG electrode 10 is impacted by an external force, to prevent the metal probe 26 from piercing the scalp and hurting a user.

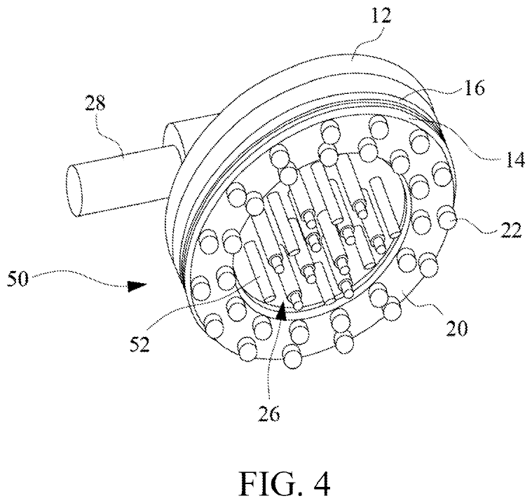

FIG. 4 is an oblique view of a dry EEG electrode according to a second embodiment of the present invention. The structural difference between a dry EEG electrode 50 according to the second embodiment and the dry EEG electrode 10 according to the first embodiment is that the dry EEG electrode 50 has a plurality of second protective protrusions 52 alternately distributed with the metal probes 26 disposed on the other plane of the metal electrode 16, and ends of the plurality of second protective protrusions 52 are flush with the ends of the plurality of first protective protrusions 22. The plurality of second protective protrusions 52 and the plurality of first protective protrusions 22 are distributed in a comb shape, for smoothly passing through the hair, and for the dry EEG electrode 50 to conform to the scalp without difficulties caused by the thickness or density of the hair.

In addition, the multi-protective protrusion comb-shaped design disperses pressure and provides appropriate support so that even if the dry EEG electrode 50 is impacted by an external force, the metal probe 26 can be prevented from piercing the scalp and hurting the user.

Descriptions of components in the dry EEG electrode 50 of the second embodiment having the same structures, component signs, and functions as those in the dry EEG electrode 10 of the first embodiment are omitted herein.

The present invention provides a dry EEG electrode, where a protection structure used in combination with a metal probe is adopted. The protection structure can protect an organism from being hurt by a metal probe 26 piercing the scalp during dry EEG electrode measurement on the organism even if the dry EEG electrode 10 is impacted by an external force.

Although the present invention has been described above with reference to preferred specific embodiments and exemplary accompanying drawings, the preferred specific embodiments and exemplary accompanying drawings should not be considered as limitations. Various modifications, omissions, and variations of the forms and the content of specific embodiments of the present invention made by a person skilled in the art should not depart from the scope claimed by the claims of the present invention.

What is claimed is:

1. A dry electroencephalographic (EEG) electrode, comprising:
a sleeve with a hollow center;
an electrode protection member, being hollow annular, wherein a plurality of first protective protrusions distributed in a comb shape are disposed on an annular plane of the electrode protection member; and
a metal electrode, sandwiched between the sleeve and the electrode protection member, comprising:
an electrode connector, formed on and electrically connected to one plane of the metal electrode, and disposed in the hollow center of the sleeve; and
a plurality of metal probes, distributed in a comb shape and electrically connected to another plane of the metal electrode, wherein each metal probe comprises a probe fixing portion, a probe extension portion, and a spring, wherein the probe fixing portion is disposed on the other plane of the metal electrode, the spring is disposed in a hollow interior of the probe fixing portion, the probe extension portion is disposed in the hollow interior of the probe fixing portion, the probe extension portion squeezes the spring, and the spring produces a reaction force on the probe extension portion, which enables the probe extension portion to extend and contract relative to the probe fixing portion, wherein
in a case that the dry EEG electrode is not pressed, ends of the probe extension portions of the plurality of metal probes protrude further relative to ends of the plurality of first protective protrusions of the electrode protection member, and in a case that the dry EEG electrode is pressed, the ends of the probe extension portions of the plurality of metal probes are flush with the ends of the plurality of first protective protrusions of the electrode protection member, and the plurality of first protective protrusions of the electrode protection member completely surround the plurality of metal probes.

2. The dry EEG electrode according to claim 1, wherein the electrode connector of the metal electrode is electrically connected to a power connector in a magnetic attraction manner, so that the dry EEG electrode rotates in a horizontal direction relative to the power connector.

3. The dry EEG electrode according to claim 1, wherein the sleeve and the electrode protection member are bonded with an adhesive, fastened with a screw, or engaged and fixed with a latch.

4. The dry EEG electrode according to claim 1, wherein a plurality of second protective protrusions alternately distributed with the metal probes are disposed on the other plane of the metal electrode, and ends of the plurality of second protective protrusions are flush with the ends of the plurality of first protective protrusions.

5. The dry EEG electrode according to claim 1, wherein the electrode protection member is made from rubber, biocompatible plastics, or an insulation material.
